(19) United States
(12) Patent Application Publication
(10) Pub. No.: US 2025/0032991 A1
(43) Pub. Date: Jan. 30, 2025

(54) HOLLOW-FIBRE MEMBRANE FILTER

(71) Applicant: FRESENIUS MEDICAL CARE DEUTSCHLAND GMBH, Bad Homburg (DE)

(72) Inventors: Michael AMES, Bad Homburg (DE); Dietmar HANSEL, Bad Homburg (DE)

(73) Assignee: FRESENIUS MEDICAL CARE DEUTSCHLAND GMBH, Bad Homburg (DE)

(21) Appl. No.: 18/714,098

(22) PCT Filed: Dec. 16, 2022
(86) PCT No.: PCT/EP2022/086315

§ 371 Date: May 29, 2024

(30) Foreign Application Priority Data
Dec. 17, 2021 (DE) 10 2021 214 636.8

(51) Int. Cl. B01D 63/02

(52) U.S. Cl. CPC B01D 63/022; B01D 2313/041; B01D 2313/205; B01D 2313/21

(57) ABSTRACT

The invention relates to a hollow fiber membrane filter comprising a plurality of hollow fiber membranes and at least two support rings, wherein the hollow fiber membranes are each enclosed by a respective support ring in a respective end portion of the hollow fiber membrane filter and are cast in a potting mass, wherein the respective support rings have a circumferential projection arranged at the upper edge which projects beyond the outer side of the circumferential side wall of a respective support ring, and wherein the circumferential projection rests on the respective terminal edges of the first and second end portions of the cylindrical housing of the hollow fiber membrane filter.

The present application relates to a hollow fiber membrane filter, in particular a hollow fiber membrane filter for extracorporeal blood treatment.

In the production of hollow fiber membrane filters, a bundle of hollow fiber membranes previously produced in a spinning and bundling process is inserted into a housing of a hollow fiber membrane filter. Almost exclusively, cylindrical housings are used as housing types. The ends of the hollow fiber membranes are potted in the end portions of the hollow fiber membrane filter with a potting mass, usually polyurethane, and are thereby fixed in the housing. The end regions of these potting masses are milled or cut away to expose the open ends of the hollow fiber membranes. End caps are then placed on the end areas of the cylindrical housing. Two flow spaces are thus formed in the hollow fiber membrane filter. With respectively one liquid port on each of the end caps and two liquid ports on the cylindrical housing, the fiber interior can be flowed through with a first liquid, e.g. Blood of a patient, and the interior space surrounding the hollow fiber membranes can be flowed through with a second liquid, e.g. dialysis fluid. For the use of such hollow fiber membrane filters in extracorporeal blood treatment or in bio-pharmaceutical processes, it is necessary to deliver such hollow fiber membrane filters in a sterile condition.

A widely used sterilization process is the steam sterilization process. After manufacture, the entire hollow fiber membrane filter is flowed through with sterilizing water vapor in multiple steps via the liquid connections. The sterilizing steam is introduced into the hollow fiber membrane filter at temperatures above 100° C. and atmospheric overpressure. Due to the different materials used in the manufacture of the hollow fiber membrane filter, the individual components of the hollow fiber membrane filter expand at different rates. Plastics consisting of polyethylene, polypropylene, polyester such as PET or PBT, polymethyl methacrylate, polystyrene or polycarbonate are commonly used for the housing of a hollow fiber membrane filter. Polyurethane is usually used for the potting material. Polymers of polysulfones and polyvinylpyrrolidones are predominantly used as materials for the hollow fiber membranes. Significant material strain can therefore occur within the hollow fiber membrane filter during the steam sterilization process.

Therefore, after steam sterilization of hollow fiber membrane filters, fractures in the potting mass are often observed. These fractures are due to the different expansion behavior of the housing, potting mass, and hollow fiber membranes. These defective hollow fiber membrane filters are detected and are discarded in the quality control procedures of industrial hollow fiber membrane manufacturing processes. Such defective hollow fiber membrane filters are undesirable. On the one hand, such defective productions result in higher production costs. Furthermore, the process flow in the industrial production of hollow fiber membrane filters is also disturbed, which leads to production delays and thus also to higher production costs.

WO 01/60502 A1 describes a hollow fiber membrane filter in which the hollow fiber membranes are essentially potted into one support ring only. The support ring has several lugs, bars and shoulders at one end. The hollow fiber membrane bundle is only connected to the support ring via the potting mass, but not to the housing of the hollow fiber membrane filter. The potting mass must be inside the support ring to prevent the support ring from bonding to the housing. From the design of the hollow fiber membrane filter shown in WO 01/60502 A1, it can be deduced that this last-mentioned requirement is demanding in terms of process technology and is therefore difficult to implement in the mass production of hollow fiber membrane filters, resp. this means that this process step may be error prone.

### OBJECT

The object therefore was to provide a hollow-fiber membrane filter that is resistant to material expansions and material strain occurring in the steam sterilization process, compared with prior art membrane filters, but which is also comparatively simple in its production.

### SUMMARY

The object is solved by a hollow fiber membrane filter having the features of claim 1. Claims 2 to 11 represent preferred embodiments.

Furthermore, the object is solved by a manufacturing process according to claim 12.

Furthermore, the object is solved by using a support ring according to claims 13 to 15.

### BRIEF DESCRIPTION OF THE FIGURES

It schematically shown in:

FIG. 1 a section of an end portion of a hollow fiber membrane filter in cross section,

FIG. 2 an end portion of a cylindrical housing of a hollow fiber membrane filter with a support ring disposed therein,

FIG. 3a an illustration of a support ring in a side view,

FIG. 3b a dimensioned illustration of a support ring with a conical inner surface of the circumferential side wall in cross-sectional view,

FIG. 3c a dimensioned illustration of a projection of the support ring with a groove in a cross-sectional view,

FIG. 4a a dimensioned illustration of a support ring with a conically shaped zone I and a cylindrically shaped zone II on the inside of the circumferential side wall in a side view,

FIG. 4b a dimensioned illustration of a support ring having a conically shaped zone I and a cylindrically shaped zone II on the inside of the circumferential side wall in a cross-sectional view, wherein the edges of the support ring present on the inside of the circumferential side wall are rounded,

FIG. 5a a dimensioned illustration of a support ring with a tapered inner side of the circumferential side wall and an undercut at the transition from its circumferential side wall to the projection on the outer side of the support ring in cross-sectional view,

FIG. 5b a dimensioned illustration of a projection of the support ring with groove and undercut in a cross-sectional view.

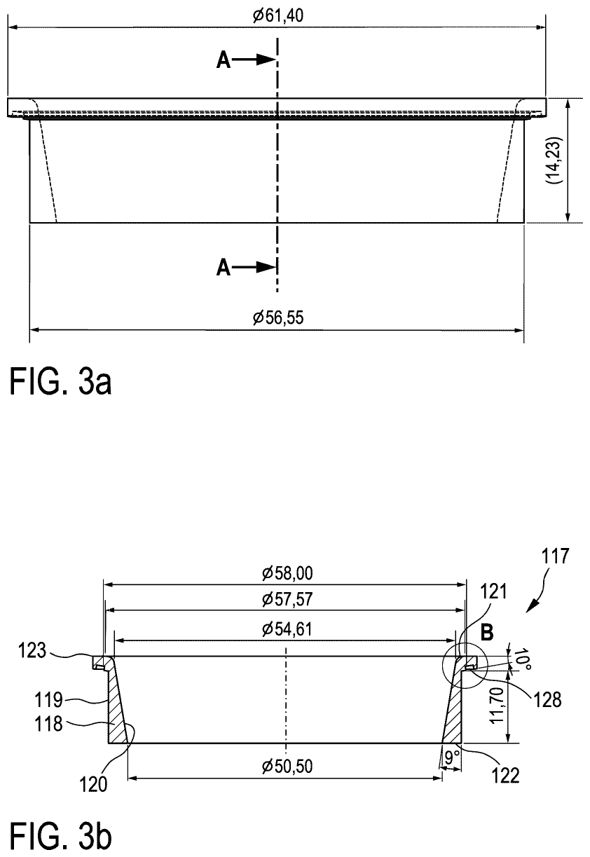

The dimensioning data in FIGS. 3a to 5b refer to the unit millimeter.

### DETAILED DESCRIPTION

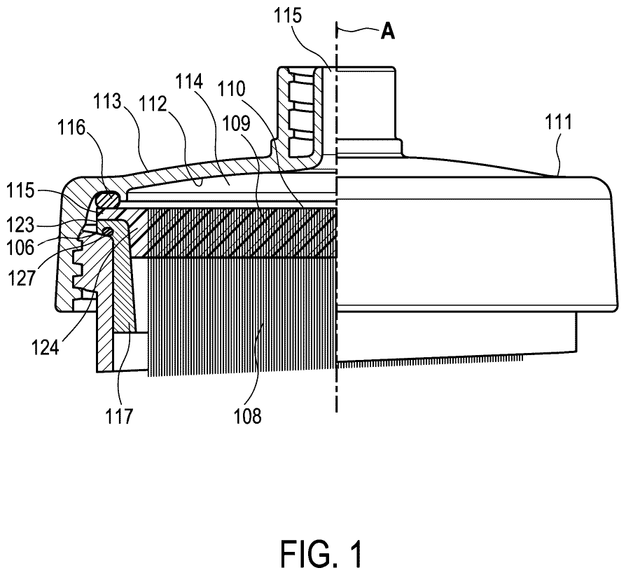

In the following, the invention is described with reference to FIGS. 1 to 5b. In a first aspect, the invention relates to a hollow fiber membrane filter comprising, a cylindrical housing 101 extending longitudinally along a central axis A having an inner side 102, an outer side 103, a housing interior 104, a first end portion 105 having a first terminal edge 106, and a second end portion having a second terminal edge, at least a first fluid port 107 disposed at the first end portion of the cylindrical housing, and optionally a second fluid port disposed at the second end portion of the cylindrical housing, a plurality of hollow fiber membranes 108 arranged in the cylindrical housing 101 and sealingly embedded in a respective potting mass 109 in the first end portion 105 and the second end portion of the cylindrical housing 101, the ends 110 of the hollow fiber membranes 108 being open so that the lumens of the hollow fiber membranes form a first flow space and the housing interior 104 surrounding the hollow fiber membranes forms a second flow space, a first and a second end cap 111 having an inner side and an outer side, the end caps being respectively side connected to the first 105 and the second end portion of the cylindrical housing 101 so as to form respective inflow or outflow chambers 114 in fluid communication with the first flow space of the hollow fiber membrane filter, the respective first and second end caps 111 or the cylindrical housing having respective third and fourth fluid access ports 115 for supplying or discharging fluid to/from the first inflow or outflow chambers 114, at least a first 116 and a second sealing ring, each sealingly arranged between a respective potting mass 109 and the inner side 112 of a respective end cap 111, which laterally seals the respective inflow and outflow chambers 114, at least a first and a second support ring 117 comprising a circumferential side wall 118 having an outer side 119 and an inner side 120, an upper edge 121 and a lower edge 122, the support rings being arranged in each of the first and second end portions 105 of the cylindrical housing such that the outer side 119 of a respective support ring 117 faces the inner side of the cylindrical housing 102 in a respective end portion 105, characterized in that the respective support rings 117 have a circumferential projection 123 arranged at the upper edge 121 and projecting beyond the outer side 119 of the circumferential side wall 118 of a respective support ring 117, wherein the circumferential projection 123 rests on the respective terminal edges 106 of the first and second end portions 105 of the cylindrical housing 101, and the respective potting masses 109 are arranged in a fluid-tight manner in an edge area 124, at least in part, between the circumferential projection 123 of a respective support ring 117 and the sealing ring.

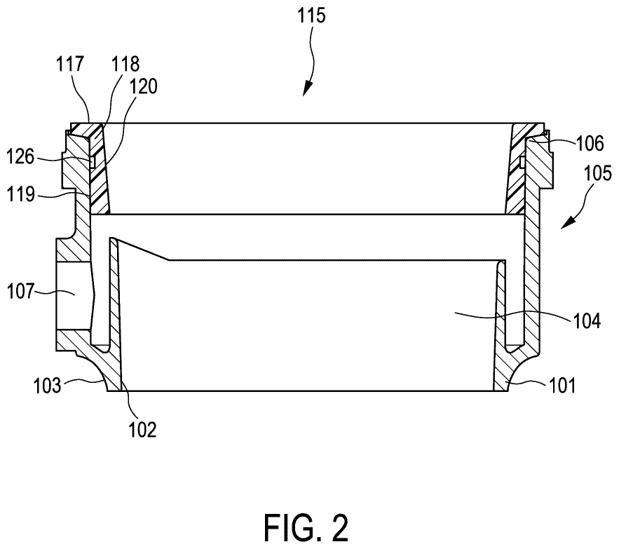

The features of this embodiment are shown in FIGS. 1 and 2. In this context, FIGS. 1. and 2 only show a section of one of the two end portions of the hollow fiber membrane filter. FIG. 2 shows, in a schematic representation, only one of the two end portions of a cylindrical housing with a support ring arranged therein.

In a hollow fiber membrane filter of the type described above, the expansion of the cylindrical housing during steam sterilization does not directly affect the potting mass, since the support ring is interposed between the terminal edge of the cylindrical housing and the potting mass. This minimizes the transfer of material stresses in the potting mass due to thermal expansion during steam sterilization. In particular, this is also due to the fact that the potting mass is not cast with parts of the cylindrical housing. The ends of the hollow fiber membranes are only potted in a potting mass in the respective support rings. The support ring itself is not firmly attached to the cylindrical housing but is merely pressed onto the terminal edge of the cylindrical housing via the end cap, the sealing ring, and the potting mass. In addition, the hollow fiber membrane filter as previously described has the advantage that the potting in the manufacture of the hollow fiber membrane filter can be carried out according to conventional proven methods, e.g. according to the process as described in EP 2 024 067 A1, and no further potting process steps need to be developed to pot the hollow fiber membranes in the support ring. This is a result from the constructional design of the support ring. The projection of the support ring covers the terminal edge of the cylindrical housing to such an extent that the potting is barred via the projection of the support ring from contacting the cylindrical housing.

In one embodiment, the hollow fiber membrane filter may be configured as a dialyzer. Within the context of the present application, the term “dialyzer” is used to represent blood filter devices used in extracorporeal blood treatment. These can be e.g. dialysis filters, hemofilters or plasma separation filters. In other applications, the hollow fiber membrane filter according to the invention can also be used as a filter for water treatment.

The term “end portion of the cylindrical housing” in the context of the present application means a portion on the cylindrical housing extending from the end of the housing toward the middle of the cylindrical housing. The term “end portion” indicates that it is a portion on the cylindrical housing that adopts only a small portion seen relatively to the longitudinal extent of the cylindrical housing. In particular, each of these end portions adopts less than one-fifth, or less than one-eighth, or less than one-tenth, or less than one-fifteenth of the total length of the cylindrical housing.

In a part of the end portion of the cylindrical housing there is the potting zone. In the context of the present application, the term “potting zone” refers to a portion in which the hollow fiber membranes of the hollow fiber membrane filter are embedded in a potting mass. The hollow fiber membranes are embedded in the potting mass in such a way that they are fixed in the support ring. The potting mass seals with the support ring. In particular, it is envisaged that the potting zone adopts less than three quarters, or less than two thirds, or less than half of the width of the support ring.

Adjacent to the potting zones on the face side, the end caps at the end of the cylindrical housing form inflow or outflow chambers. In the context of the present application, the term “inflow or outflow chamber” refers to a volumetric region in the hollow fiber membrane filter in which fluid can enter, either before it enters the first flow space of the hollow fiber membrane filter or after it exits the first flow space of the hollow fiber membrane filter. The first inflow and outflow chambers sealingly connect to the potting zone and/or to the end of the end section of the cylindrical housing via the sealing rings. The first inflow or outflow chambers each include a first fluid port for introducing or discharging fluid into/from the first inflow or outflow chambers. The first inlet or outlet chambers are therefore in fluid communication with the first flow space of the hollow fiber membrane filter, which is formed by the lumens of the hollow fiber membranes. In the context of the present application, “lumina” or “lumen” is understood to mean the cavity of the hollow fiber membranes.

The term “sealing ring” is understood to mean a liquid-tight seal that is arranged circumferentially or in ring shape. According to the hollow fiber membrane filter described above, the sealing ring is located between the inside of the end cap and the potting mass. An appropriately designed sealing ring can be designed as an O-ring and e.g. consist of an elastomeric material, such as a silicone rubber.

In the context of the present application, the term “support ring” is understood to mean a sleeve-shaped component consisting essentially of a circumferential side wall. The support ring is suitable for holding the hollow fiber membranes and the potting mass. The support ring is advantageously made of a plastic material, such as e.g. polyethylene, polypropylene, polyester, polymethylmethacrylate, polystyrene or polycarbonate. Polypropylene is preferred. The support ring has an upper and a lower edge. The upper edge is understood to be the closing edge in the direction of a respective end cap. The lower edge is understood to be the closing edge of the support ring towards the middle area of the hollow fiber membrane filter. At the upper edge of the support ring is a circumferential projection configured to project the circumferential side wall of the support ring on the outside of the support ring. The projection can be flange-like and right-angled to the central axis of the cylindrical housing. However, it is preferred that the projection deviating therefrom adopts an inclined angle to allow better centering of the support ring in the end portion of the cylindrical housing, further also to allow the support ring to adopt a low-movement fit in the end portion of the cylindrical housing. The flange-like projection may abut the circumferential side wall of the support ring at an angle of 90 to 70° relative to the center axis of the cylindrical housing.

A further embodiment of the first aspect is characterized in that the potting mass 109 has, in its edge area 123, the shape of a flange 125 which rests on the projection 123 of the support ring 117 and is arranged between the potting mass 109 and the sealing ring 116. The individual features of this embodiment are shown in FIG. 1. In this embodiment, not the entire layer thickness of the potting mass with which the ends of the hollow fiber membrane are potted is not located between the projection of the support ring and the sealing ring. According to this embodiment, part of the potting mass can be saved. In addition, this embodiment allows more flexible fit of the potting mass within the support ring, so that any material strain that occur can be compensated for more effectively.

A further embodiment of the first aspect is characterized in that a respective radially sealing sealing ring is disposed between the outer surface 119, the circumferential side wall 118 of a respective support ring 117 and the inner surface 102 of the cylindrical housing in a respective end portion 105. In FIG. 2, a circumferential groove 126 is shown, which is intended to receive the radially sealing sealing ring and which acts in a sealing manner with respect to the cylindrical housing. In one aspect, the radially sealing sealing ring prevents leakage between the second flow space from the housing interior 104 between the support ring 117 and the inner surface 102 of the cylindrical housing 101 in the end portion 105. In addition, the radially sealing sealing ring also allows the support ring to be mounted flexibly to a certain extent in the end portion of the cylindrical housing, so that further material strain can be minimized during steam sterilization. The radially sealing sealing ring can be made of an elastomeric plastic material, e.g. made of a silicone rubber.

Another embodiment of the first aspect is characterized in that the support ring 117 has on the outer side 119 of the circumferential side wall 118 facing the inner side 102 of the cylindrical housing 101 in a respective end potion 105, a circumferential groove 126 in which the radially sealing sealing ring is at least partially recessed. This embodiment is shown in part in FIG. 2 shown without the radially sealing ring. The groove causes the sealing ring to be held in position between the inside 102 of the cylindrical housing 101 and the outside 119 of the circumferential side wall 118 of the support ring 117.

A further embodiment of the first aspect is characterized in that an axially sealing sealing ring 127 is arranged between the projection 123 of a respective support ring 117 and the respective terminating edge 106 of a respective end portion 105, respectively. This embodiment is shown in FIG. 1. The axial sealing sealing ring 127, when in position, provides an improved sealing action between the outer surface 119 of the support ring 117 and the inner surface 102 of the cylindrical housing 101 in the end portion 105. In addition, the axial sealing sealing ring 127 also improves the bedding of the support ring 117 in the seat of the end portion 105 of the cylindrical housing and allows a low possible mobility of the support ring 117 in the end portion 105 of the cylindrical housing 101 under the conditions of steam sterilization. In one embodiment, the axially sealing sealing ring may be arranged on the support ring 117 in combination with the radially sealing support ring described previously.

In another embodiment of the first aspect, the support ring 117 has on the side of the projection 123 facing the terminating edge 126 of a respective end portion 105, a circumferential groove 128 into which the radially sealing ring 127 is at least partially recessed. Groove 128 is shown in FIGS. 3b and 3c. 3c. The groove holds the radially sealing sealing ring in position, which is particularly advantageous when manufacturing a hollow fiber membrane filter according to this embodiment.

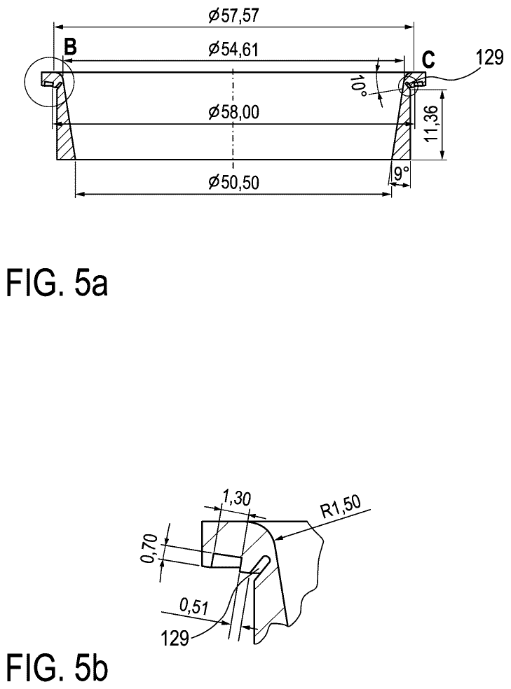

A further embodiment of the first aspect is characterized in that the support ring 117 has a circumferential undercut 129 at the transition from its circumferential side wall 118 to the projection 123 on the outer side 119. This embodiment is illustrated in FIGS. 5a and 5b. The transition from the circumferential side wall 118 to the projection 123 on the outside 119 of the support ring 117 forms an inner edge. In the context of the present application, the term “undercut” is understood to mean an ablation at the rotationally symmetrical inner edge of the support ring. According to FIGS. 5a and 5b, the wall thickness of the circumferential side wall 118 of the support ring is significantly reduced by the undercut. Therefore, the projection 123 can flex to a small extent relative to the circumferential sidewall of the support ring. Possible material strain that can occur during steam sterilization can thus be compensated for in an improved manner.

Another embodiment of the first aspect is characterized in that the support ring 117 has a height in the direction of the longitudinal orientation A of the hollow fiber membrane filter of 2 to 10%, preferably 2 to 9%, more preferably 3 to 8%, more preferably 4 to 7% of the total length of the hollow fiber membranes 108. The height of an exemplary embodiment of a support ring 117 is shown in the dimensioned illustration of FIG. 3a. In the context of the present invention, the height of a support ring is understood to be the distance from the upper edge 121 to the lower edge 122 of a support ring. In commercially available hollow fiber membrane filters used for extracorporeal blood treatment, the length of the hollow fiber membrane is about 235 mm. The height of the support rings can be within the previously mentioned ranges and can be varied depending on the number of hollow fiber membranes. Depending on the height of the potting mass, the height of the support ring also provides lateral support for the hollow fiber membrane bundle in the hollow fiber membrane filter. In particular, it is provided that the height of the potting mass in the support ring is only ¼, preferably ⅓, and less than half of the height of the support ring.

A further embodiment of the first aspect is characterized in that the circumferential side wall 118 of the support rings 117 on the respective inner side 120 of the support rings are shaped conical at least in sections from the upper edge 121 toward the lower edge 122. The conical shape of the inner surface 120 of the support ring 117 is shown in FIGS. 1, 2, 3a, 3b, 4a, 4b, and 5a. The conical shape allows for improved containment of the hollow fiber membrane bundle and potting of the ends of the hollow fiber membranes in the support ring 117.

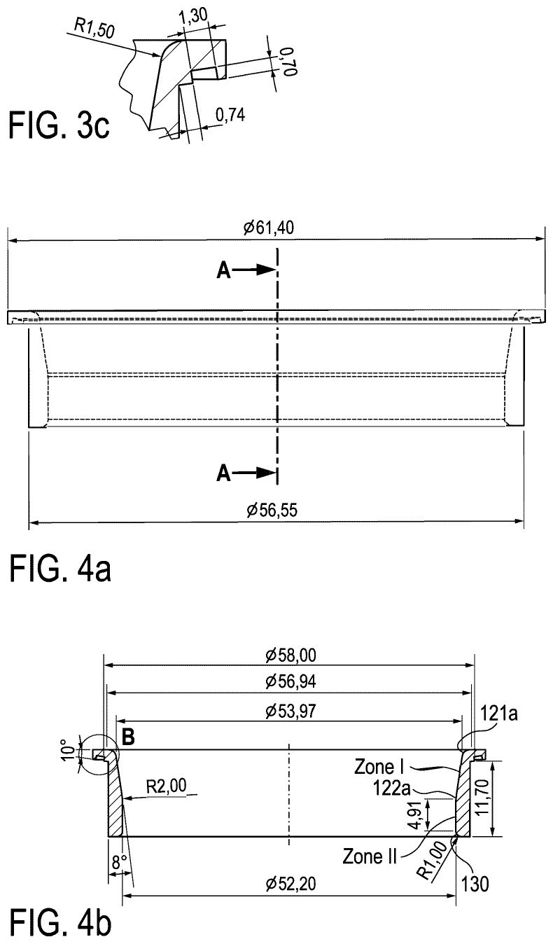

In certain embodiments, the circumferential side wall (118) of the support rings on the inner side (120) has at least zones I and II, of which zone I and/or II is/are conically shaped or at least zone I or II is/are cylindrically shaped and zone II or I is/are conically shaped. One such embodiment of the support ring is shown in FIG. 4b shown. According to FIG. 4b, the support ring 117 has on the inner side 120 of the circumferential side wall 118, a zone I adjacent to the upper edge 121 of the circumferential side wall 118 and a zone II adjacent to the lower edge 122 of the support ring. In the zone I region, the inner surface 120 of the circumferential side wall 118 has a conical shape; in the zone II region, the inner surface 120 of the circumferential side wall has a cylindrical shape. In common embodiments, the outer surface 119 of the circumferential side wall 118 is thereby cylindrically shaped over the entire height up to the projection 123.

In another detail of the previously described embodiment, the at least one conically shaped zone I or II or the entire conical shape of the inner surface 119 of the circumferential side wall 118 relative to the direction of the central axis A assumes a cone angle of 3 to 15 degrees, preferably 4 to 12 degrees, more preferably 5 to 11 degrees, further preferably 6 to 10 degrees. Such embodiments are shown in FIGS. 3b, 4b, 5a shown.

In another detail of the embodiment previously described, at least the edges 121a, 122a, 130 of the support ring 117 present on the inner side 119 of the circumferential side wall 118 are rounded. A corresponding embodiment is illustrated in FIG. 4b shown. The rounding of the edges prevents damage to the hollow fiber membranes that may contact the inside 119 of the circumferential side wall 118 of the support ring.

In a second aspect, the invention relates to the manufacture of a hollow fiber membrane filter according to the features of one embodiment of the first aspect, comprising the steps of providing a cylindrical housing 101 extending longitudinally along a central axis A having an inner side 102, an outer side 103, a housing interior 104, a first end portion 105 having a first terminal edge 106, and a second end portion having a second terminal edge, providing two support rings 117, each having a circumferential side wall 118 with an outer side 119 and an inner side 120, an upper edge 121 and a lower edge 122, and a projection 123 located at the upper edge 121 of a respective support ring 117 and projecting beyond the outer side 119 of the circumferential side wall 118 of a respective support ring 117, inserting a support ring 117 in each of the respective end portions 105 of the cylindrical housing 101, such, that the support rings 117 are arranged in each of the first and second end portions 105 of the cylindrical housing and the outer side 119 of a respective support ring 117 faces the inner side 102 of the cylindrical housing in a respective end portion 105 and the respective support rings 117 rest with the circumferential projection 123 arranged at the upper edge 121 on the respective closing edges 106 of the first and second end portions 105 of the cylindrical housing 101, inserting a hollow fiber membrane bundle consisting of a plurality of hollow fiber membranes into the cylindrical housing 101 and into the respective support rings, potting the ends of the hollow fiber membranes with the respective support rings 117 in a potting zone with a potting mass 109 and exposing the ends of the hollow fiber membranes, mounting end caps 111 on the respective end portions 105 of the cylindrical housing 101 while inserting a sealing ring 116 between the inside 112 of the end cap 111 and in an edge area 124 of the potting mass 109.

In this context, “edge area of the potting mass” is understood to mean a circumferential annular part of the potting mass that is adjacent to the support ring but in which no hollow fiber membranes are potted.

Exposing the ends of the hollow fiber membranes can be accomplished by known methods, such as. milling off or cutting off part of the potting mass on the face side.

In accordance with the method described herein, a hollow fiber membrane filter is manufactured that is designed to reduce material stresses within the hollow fiber membrane filter during the process of steam sterilization. The process also has the advantage that previously existing processes for manufacturing hollow fiber membrane filters do not have to be significantly modified. Essentially, the manufacturing process requires that during potting, the potting mass is substantially not in contact with the cylindrical housing so that the hollow fiber membranes in the potting mass remain decoupled from the cylindrical housing. In the potting process step, a method can be used, for example, that. described in EP 2 024 067 A1. According to this method, potting caps are placed on the end portions of the hollow fiber membrane filter and the liquid potting mass is introduced into the end portion of the hollow fiber membrane filter so that hollow fiber membranes are embedded into a potting zone in the support ring. Since the projection of the support ring rests on the terminal edge of the cylindrical housing, it is essentially avoided that the potting mass can contact the cylindrical housing. “Substantially” in this context means that the potting mass cannot form a firm bond with the cylindrical housing and support ring, so the support ring is not bonded in the end portion of the cylindrical housing. After curing of the potting mass, the hollow fiber membranes are fixed inside the support ring in the potting mass.

To increase adhesion between the support ring and the potting mass, the support ring can be pretreated. In particular, the surface of the inner surface 120 of the circumferential side wall 118 of the support ring 117 may be modified with, for example, plasma treatment or corona treatment so that adhesion of the potting mass to the support ring is improved. In these treatment processes, the surface of the treated support ring is hydrophilic modified, enabling increased adhesion of the potting mass. In particular, the afore-mentioned surface treatments produce chemically hydrophilic groups, such as hydroxy- or carboxyl-groups, so that a chemical reaction between the potting mass and the surface is enabled.

The method described above may include further process steps necessary for manufacturing a hollow fiber membrane filter according to an embodiment according to the first aspect. In particular, in further procedural steps, e.g. the axially and/or radially sealing sealing rings are placed into the position provided for this purpose on the outside of the support ring.

In a third aspect, the invention relates to the use of a support ring 117 comprising a circumferential side wall 118 having an outer surface 119, an inner surface (120), upper 121 and lower 122 edges, and a circumferential projection 123 disposed at the upper edge 121 projecting the outer surface 119 of the circumferential side wall 118 for the construction of a hollow fiber membrane filter. Preferably, a support ring is used which additionally has a circumferential groove 126, 128 on the outer side 119 of the circumferential side wall 118, or on the projection 123, for receiving a radially or axially sealing sealing ring. Further preferably, a support ring is used wherein the support ring is formed on the inner side 120 of the circumferential side wall 118 from the upper edge 121 in the direction of the lower edge 122 at least in sections in a conical shape.

### EXAMPLE

Example: Production of a hollow fiber membrane filter according to the invention. In a commercially available cylindrical filter housing of the type HF80S from Fresenius Medical Care Deutschland GmbH, a support ring was inserted at each end portions of the cylindrical housing as shown in FIG. 3b with an axially sealing sealing ring recessed in the groove running circumferentially along the projection. The cylindrical housing was made of polycarbonate, and the support ring was made of a polypropylene material. The support ring had the dimensions shown in FIG. 3b (in mm). The surface of the inner side of the support ring was chemically modified by corona treatment. A hollow fiber membrane bundle wrapped in an envelope film enclosing approximately 12300 hollow fiber membranes, each with an inner diameter of 200 μm, a wall thickness of 40 μm, having a double wave-shaped texture with wavelengths of 30 and 3 mm, manufactured as described in Example 1 of EP 3 423 173 A1, were inserted into the cylindrical housing through the support rings. The further production of the hollow fiber membrane filter was carried out according to known methods and process steps. 109 g of polyurethane per filter was used for potting, and the active length of a hollow fiber (length of the hollow fiber membrane between the potting ends) was 230 mm. A hollow fiber membrane filter was obtained, which in its construction of the respective end portions corresponds the filter shown in FIG. 1. A hollow fiber membrane filter was obtained, which in its construction of the respective end portions corresponds the filter shown in FIG. 1.

### Comparative Example

The filter of the comparison example was manufactured in the same way as the filter described above, but without the installation of the support rings. The hollow fiber membranes were cast directly in the filter housing using conventional methods.

### Thermal Stress Test

A hollow fiber membrane filter manufactured according to the working example described above and a hollow fiber membrane filter manufactured according to the comparative example are subjected to a thermal stress test.
Step 1: For this purpose, the respective hollow fiber membrane filter is connected to a media supply apparatus on the blood side and on the dialysate side via respective two connections in a vertical orientation. The hollow fiber membrane filter is treated from the upper side with pure steam and an absolute pressure of 2.35 bar at a temperature of 125° C. for a time of 17 min. Exposure time is recorded with application of the pure steam.
Step 2: After exposure to steam in step 1, exposure is switched to ultrapure water (HPW), setting the ultrapure water to a temperature of 95° C. Blood and dialysate side are exposed with an absolute pressure of 3.2 bar for a time of 5 min 20 sec. Exposure time is recorded with application of the ultrapure water.
Step 3: After this step, the dialysate side is emptied and is then filled with sterile air at an absolute pressure of 2.8 bar. The water on the blood side is cooled to 54° C.; it is observed whether air passes into the water on the blood side. For this purpose, a sight glass of an outer diameter of 15 mm and an inner diameter of 10 mm with a sampling length of 65 mm is installed in the water supply. The sight glass is monitored with a digital camera and the number of bubbles, the size of each bubble and the size of all bubbles are calculated. The procedure is known as “bubble test”. Measuring was done for 15 sec. The experiment is stopped if the number of bubbles of 7 with an area observed via the digital camera of at least 1.7 mm², or one bubble of an observed area greater than 17 mm², or the total observed area of all detected bubbles exceeds 34 mm². Measurement is performed in 4 times one after the other, wherein the hollow fiber membrane filter is evaluated to be leaking if one of the afore-mentioned end-point criteria is reached once.
Step 4: After the bubble test, sterile dry air is applied at a temperature of 110° C. at an absolute pressure of 1.7 bar on the blood side and 1.5 bar on the dialysate side for 25 min.

If a bubble test as described above does not show any leakage, the test is counted as one cycle. A hollow fiber membrane filter that is leaky from the start would thus reach cycle number 0. A hollow fiber membrane filter detected as defective in the third cycle would thus be assigned the cycle number 2.

4 hollow fiber membrane filters were tested according to Example 1. The cycle numbers obtained were: 30, 30, 13, 30. After 30 successful cycles, the experiment was terminated.

6 hollow fiber membrane filters of the comparative example were tested. The determined cycle numbers were: 8, 8, 2, 2, 2, 8.

As shown, embodiments according to the invention are considerably more resilient than embodiments that do not have the features of the invention.

1. A hollow fiber membrane filter comprising
a cylindrical housing extending longitudinally along a central axis having an inner side, an outer side, a housing interior, a first end portion having a first terminal edge, and a second end portion having a second terminal edge,
at least a first fluid port disposed at the first end portion of the cylindrical housing, and optionally a second fluid port disposed at the second end portion of the cylindrical housing,
a plurality of hollow fiber membranes arranged in the cylindrical housing and sealingly embedded in a respective potting mass in the first end portion and the second end portion of the cylindrical housing, the ends of the hollow fiber membranes being open so that the lumens of the hollow fiber membranes form a first flow space and the housing interior surrounding the hollow fiber membranes forms a second flow space,
a first and a second end cap having an inner side and an outer side, the end caps being respectively face side connected to the first and the second end portion of the cylindrical housing so as to form respective inflow or outflow chambers in fluid communication with the first flow space of the hollow fiber membrane filter, the respective first and second end caps or the cylindrical housing having respective third and fourth fluid access ports for supplying or discharging fluid to/from the first inflow or outflow chambers,
at least a first and a second sealing ring, each sealingly arranged between a respective potting compound and the inner side of a respective end cap, which laterally seals the respective inflow and outflow chambers,
at least a first and a second support ring comprising a circumferential side wall having an outer side and an inner side, an upper edge and a lower edge, the support rings being arranged in each of the first and second end portions of the cylindrical housing such that the outer side of a respective support ring faces the inner side of the cylindrical housing in a respective end portion, wherein
the respective support rings have a circumferential projection arranged at the upper edge and projecting beyond the outer side of the circumferential side wall of a respective support ring, wherein the circumferential projection rests on the respective terminal edges of the first and second end portions of the cylindrical housing, and the respective potting masses are arranged in a fluid-tight manner in an edge area, at least in part, between the circumferential projection of a respective support ring and the sealing ring.

2. The hollow fiber membrane filter according to claim 1, wherein the potting mass has in its edge area the shape of a flange which rests on the projection of the support ring and is arranged between the potting compound and the sealing ring.

3. The hollow fiber membrane filter according to claim 1, wherein a radially sealing sealing ring is arranged between the outer side of the circumferential side wall of a respective support ring and the inner side of the cylindrical housing in a respective end portion.

4. The hollow fiber membrane filter according to claim 3, wherein the support ring on the outer side of the circumferential side wall facing the inner side of the cylindrical housing in a respective end portion has a circumferential groove into which the radially sealing sealing ring is at least partially recessed.

5. The hollow fiber membrane filter according to claim 1, wherein an axially sealing sealing ring is arranged between the projection of a respective support ring and the respective terminal edge of a respective end portion.

6. The hollow fiber membrane filter according to claim 5, wherein the support ring has, on the side of the projection facing the terminal edge of a respective end portion, a circumferential groove into which the axially sealing sealing ring is at least partially recessed.

7. The hollow fiber membrane filter according to claim 1, wherein the support ring has a circumferential undercut at the transition from its circumferential side wall to the projection on the outside.

8. The hollow fiber membrane filter according to claim 1, wherein the circumferential side wall of the support rings on the respective inner side of the support rings are shaped conical at least in sections from the upper edge toward the lower edge.

9. The hollow fiber membrane filter according to claim 8, wherein the circumferential side wall of the support rings on the inner side has at least zones I and II, of which zone I and/or II is/are conically shaped or at least zone I or II is/are cylindrically shaped and zone II or I is/are conically shaped.

10. The hollow fiber membrane filter according to claim 9, wherein the at least one conically shaped zone I or II has a cone angle of 3 to 15 degrees relative to the direction of the central axis.

11. Hollow The hollow fiber membrane filter according to claim 9, wherein at least the edges of the support ring present on the inner side of the circumferential side wall are rounded.

12. A method of making the hollow fiber membrane filter according to claim 1, comprising the steps of
providing a cylindrical housing extending longitudinally along a central axis having an inner side, an outer side, a housing interior, a first end portion having a first terminal edge, and a second end portion having a second terminal edge,
providing two support rings, each having a circumferential side wall with an outer side and an inner side, an upper edge and a lower edge, and a projection located at the upper edge of a respective support ring and projecting beyond the outer side of the circumferential side wall of a respective support ring,
inserting a support ring in each of the respective end portions of the cylindrical housing, such, that the support rings are arranged in each of the first and second end portions of the cylindrical housing and the outer side of a respective support ring faces the inner side of the cylindrical housing in a respective end portion and the respective support rings rest with the circumferential projection arranged at the upper edge on the respective terminal edges of the first and second end portions of the cylindrical housing,
inserting a hollow fiber membrane bundle consisting of a plurality of hollow fiber membranes into the cylindrical housing and into the respective support rings,
potting the ends of the hollow fiber membranes with the respective support rings in a potting zone with a potting mass,
mounting end caps on the respective end portions of the cylindrical housing while inserting a sealing ring between the inside of the end cap and an edge area of the potting mass.

13. A method for making a hollow fiber membrane, said method comprising utilizing a support ring comprising a circumferential side wall having an outer surface, an inner surface, upper and lower edges, and a circumferential projection disposed at the upper edge projecting the outer surface of the circumferential side wall.

14. The method according to claim 13, wherein the support ring has a circumferential groove on the outer side of the circumferential side wall, or on the projection.

15. The method according to claim 13, wherein the support ring is formed on the inner side of the circumferential side wall from the upper edge in the direction towards the lower edge at least in sections in a conical shape.
